(19) United States
(12) Patent Application Publication
(10) Pub. No.: US 2012/0244690 A1
(43) Pub. Date: Sep. 27, 2012

(54) ION IMPLANTED RESIST STRIP WITH SUPERACID

(75) Inventor: Yoshihiro Uozumi, Somers, NY (US)

(73) Assignee: TOSHIBA AMERICA ELECTRONIC COMPONENTS, INC., Irvine, CA (US)

(21) Appl. No.: 13/069,625

(22) Filed: Mar. 23, 2011

(51) Int. Cl. H01L 21/426; C23F 1/08; C01B 17/46; C07F 5/02; C07C 309/06; B08B 13/00; G03F 7/42

(52) U.S. Cl. 438/514; 156/345.23; 156/345.21; 134/3; 510/176; 568/5; 562/113; 423/467; 257/E21.474

(57) ABSTRACT

According to certain embodiments, a resist is placed over the surface of a semiconductor structure, wherein the resist covers a portion of the semiconductor structure. Dopants are implanted into the semiconductor structure using an ion implantation beam in regions of the semiconductor structure not covered by the resist. Due to exposure to the ion implantation beam, at least a portion of the resist is converted by exposure to the ion beam to contain an inorganic carbonized material. The semiconductor structure with resist is contacted with a superacid composition containing a superacid species to remove the resist containing inorganic carbonized materials from the semiconductor structure.

### FIELD

Embodiments described herein generally relate to methods and devices for removing carbonized materials from semiconductor structures.

### BACKGROUND

Semiconductor devices are formed by combining materials having varying conductive properties. In general, semiconductor structures and devices can contain electric insulators, electrical conductors and semiconductor materials that have electrical properties intermediate to insulators and conductors. The properties of semiconductor materials can be adjusted through the introduction of dopants or impurities.

Impurities are added to semiconductor materials using ion implantation techniques. Ion implantation techniques function through the production of ions of a desired element or molecule produced in an ion source. The ion is accelerated to a high energy using magnetic fields, where higher energy results in a greater depth of penetration. However, high-energy ion implantation can result in undesirable chemical changes to a resist used for selectively doping a semiconductor.

### BRIEF DESCRIPTION OF DRAWINGS

FIG. 1 shows an embodiment semiconductor with a resist present on a surface thereof.

FIG. 2 shows an embodiment semiconductor structure with impurity doped regions.

FIG. 3 shows Raman spectra of resists implanted with varying ion doses.

FIG. 4 shows an embodiment semiconductor structure after contact with a superacid composition in accordance with some embodiments.

FIG. 5 shows an embodiment apparatus for stripping a resist from a semiconductor structure.

FIG. 6 shows a flow chart for an exemplary methodology for removing a carbonized resist in accordance with some embodiments.

### DETAILED DESCRIPTION

According to one embodiment, a resist is placed over the surface of a semiconductor structure, wherein the resist covers a portion of the semiconductor structure. Dopants are implanted into the semiconductor structure using an ion implantation beam in regions of the semiconductor structure not covered by the resist. The resist is exposed to the ion implantation beam in the process of blocking deposition of dopants into regions of the semiconductor structure covered by the resist. Due to exposure to the ion implantation beam, at least a portion of the resist is converted by exposure to the ion beam to contain an inorganic carbonized material. The resist is contacted with a superacid composition containing a superacid species to affect the removal of the resist from the semiconductor structure.

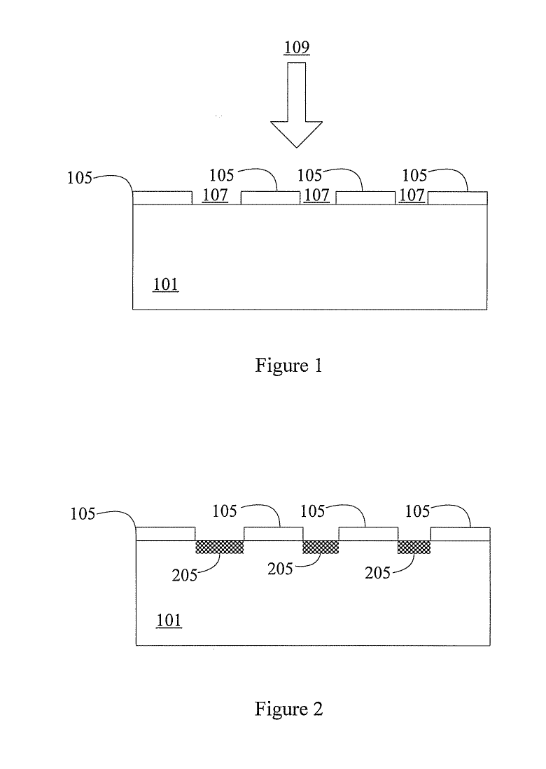

As shown in FIG. 1, a resist 105 is placed over a portion of a semiconductor structure 101. Openings 107 in the resist—regions where the resist does not cover the semiconductor structure—allow for an ion implantation beam 109 to contact the surface of the semiconductor structure 101.

As shown in FIG. 2, impurity regions 205 are formed on the semiconductor structure 101 due to exposure to the ion implantation beam 109. Such doped regions can form the source and drain regions of transistor structures or other functional regions. Before additional processing acts can be performed, the resist 105 typically needs to be removed.

The resist 105 is typically formed from an organic polymer material. The resist can contain light-sensitive materials to assist in patterning the resist; however, the methods disclosed herein are not dependent upon any specific composition for the resist. Exposure of the resist to an ion implantation beam introduces undesirable chemical changes to the resist 105 complicating removal of the resist 105. High-energy ion implantation beams can carbonize the resist. As defined herein, carbonization refers to a portion of the resist containing inorganic carbon bonds. A material containing inorganic carbon bonds has at least of a portion of the carbon atoms contained in the resist bonded only to other carbon atoms. That is, a portion of the carbon atoms in a carbonized inorganic material are not bonded to organic bases such as methyl or ethyl bases. However, it must be noted that carbon-hydrogen bonds can be present in the resist after exposure.

Carbonization is indicated by a portion of the carbon atoms present being involved only in carbon-carbon inorganic bonding. A carbonized material having inorganic carbon-carbon bonds can contain one or more of graphite or micro crystallized carbon. Graphite is an allotrope of carbon where carbon forms hexagonal rings of carbon atoms bonded to three other carbon atoms. Mirco crystallized carbon is a material that contains sp³ hybridized carbon, however, a full three-dimensional lattice is not present. As examples, the inorganic carbonized material can be one or more selected from graphite, fullerene, graphene, carbon nano-tube and micro crystallized carbon, among others.

As discussed, exposure of the organic polymer material of the resist converts at least a portion of the organic material in the resist to a carbonized inorganic material. The extent of carbonization increases as the exposure of the resist to the ion implantation beam increases. A measure of the extent of exposure of the resist is the energy of the ion implantation beam. In one embodiment, the ion implantation beam has energy from about 1 to about 1000 keV. In another embodiment, the ion implantation beam has energy from about 1 to about 100 keV. In yet another embodiment, the ion implantation beam has energy greater than about 3 keV.

Another measure of exposure of the resist to the ion implantation beam is the ion dose delivered to the exposed regions of the semiconductor structure. Although regions of the semiconductor structure covered by the resist do not receive an ion dose, the resist receives the same exposure as the regions of the semiconductor structure actually implanted with ions. In one embodiment, the semiconductor structure including a resist is exposed to an ion implantation beam such that at least one region of the semiconductor device has impurities at a concentration from about 1×10¹¹ to about 1×10¹⁷ atoms/cm². In another embodiment, the semiconductor structure including a resist is exposed to an ion implantation beam such that at least one region of the semiconductor device has impurities at a concentration from about 1×10¹² to about 1×10¹⁶ atoms/cm². In yet embodiment, the semiconductor structure including a resist is exposed to an ion implantation beam such that at least one region of the semiconductor device has impurities at a concentration more than about 1×10¹⁴ atoms/cm². Carbonization can occur regardless of the identity of the implanted ion including both p-type and n-type impurities.

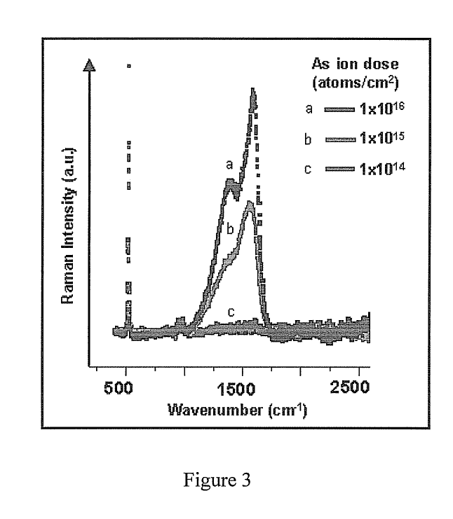

The presence of inorganic carbonized material in the resist after exposure to an ion beam can be determined and measured through the use of Raman spectroscopy. Inorganic carbonized material produces light scattering intensity at a wavenumber shift of about 1600 cm⁻¹, where organic polymer material produces minimal scattering intensity at a wavenumber shift of 1600 cm⁻¹. FIG. 3, reported by G. G. Totir et al. in ECS2007, incorporated herein by reference, shows Raman spectra obtained from a resist sensitive to deep-ultraviolet radiation implanted with different doses of As at 40 keV, as shown. The increase in Raman intensity at about 1600 cm⁻¹ indicates an increase in the amount of inorganic carbonized material after exposure to increasing doses of ions.

Inorganic carbonized materials are typically difficult to remove from the surface of the semiconductor device. Wet stripping techniques employing a mixture of sulfuric acid and hydrogen peroxide are often not able to remove all residues of carbonized material from the semiconductor structure. Further, the use of sulfuric acid and hydrogen peroxide has compatibility issues with semiconductor structures containing metal gates and/or high-k materials. In particular, the oxidizing nature of sulfuric acid and hydrogen peroxide mixture can attack and oxidize the materials forming the metal gate and/or high k-materials, including tungsten and/or titanium nitrides. Dry ashing techniques also leave residues of the inorganic carbonized material. Further, dry ashing processes generally add to the production costs of a semiconductor device compared with wet stripping techniques.

Several different kinds of metals can be incorporated into metal gate structures or other metal containing-structures. Such metals include at least one or more selected from Ti, Zr, Hf, Nb, Ta, Mo, W, Mn, Fe, Ru, Co, Ni, Pd, Pt, La, Er, Al, Ga, Ge, In, Mg, Y, and alloys thereof. As defined herein, alloys include any combination of one or more selected from the described metals with nitrogen to form a metal nitride, any combination of one or more selected from the described metals with oxygen to form a metal oxide, and any combination of one or more selected from the described metals with one or more another metals.

In embodiments disclosed herein, a resist containing inorganic carbonized material is removed through contact with a composition containing a superacid. A superacid is capable of protonating inorganic carbon leading to the breakdown of the carbonized material to smaller fragments that can be more easily dissolved and removed from the semiconductor structure.

Those skilled in the art will recognize that well-known semiconductor fabrication techniques including depositing materials, masking, photolithography, etching, and implanting are useful in forming the described devices or structures. Deposition of materials for forming semiconductor structures can be by low pressure chemical vapor deposition, chemical vapor deposition, atomic layer deposition, spin coat deposition and the like. Conserved reference numbers match like elements.

Terms, such as “on,” “above,” “below,” and “over,” used herein, are defined with respect to the plane defined by the surface of a semiconductor substrate. The terms “on,” “above,” “over,” etc. indicate that the subject element is farther away from the plane of the semiconductor substrate than another element referred to as a spatial reference. The term “below” and similar terms indicate that the subject element is closer to the plane of the semiconductor substrate than another element referred to as a spatial reference. The terms “on,” “above,” “below,” and “over,” etc. only indicate a relative spatial relationship and do not necessarily indicate that any particular elements are in physical contact. The preceding definitions apply throughout this document. As used throughout this disclosure, similar reference numbers refer to similar structures and features.

A superacid is any acidic composition having a thermodynamic activity of hydrogen ion greater than concentrated sulfuric acid. The acidity of superacids can be measured through reference to Hammett acidity function (Hₒ). In one embodiment, a superacid composition has a Hₒ less than about −10. In another embodiment, a superacid composition has a Hₒ less than about −12. In yet another embodiment, a superacid composition has a Hₒ from about −12 to about −60. In still another embodiment, a superacid composition has a Hₒ from about −12 to about −25. Superacid compositions contain one or more superacid species. As defined herein, a superacid species is a compound that has a Hammett acidity function less than about −12 when in pure form or a mixture of a Lewis acid and a Bronsted acid having a Hammett acidity function less than about −12 in pure form. Superacid compositions include compositions containing one or more of trifluoromethanesulfonic acid, a mixture of antimony pentafluoride and fluorosulfonic acid, a mixture of antimony pentafluoride and hydrofluoric acid, carborane acid, and fluorosulfonic acid.

Those skilled in the art will recognize that the exact identity of the superacid is not critical to the ability to remove carbonized material from the semiconductor structure. Rather, a superacid composition having sufficient acidity to transfer protons to an inorganic carbonized material will affect the removal of any inorganic carbonized material in a resist. Superacid compositions with a sufficiently low (i.e. negative) Hammett acidity function have a high acid activity needed to achieve a sufficiently low Hammett acidity function.

In one embodiment, a superacid composition contains about 5% or more of one or more superacid species by weight. In another embodiment, a superacid composition contains about 50% or more by weight of one or more superacid species and one or more diluents. As defined herein, a diluent is any material that is not itself a superacid species. Diluents can include both protic substances including water and alcohols, such as ethanol and methanol. Further, diluents can include organic solvents and aprotic substances such as alkanes, cycloalkanes and aromatic solvents such as benzene, toluene, diisopropylbenzene, dipropylbenzene, and diethylbenzene. In yet another embodiment, a superacid composition contains about 70% or more by weight of one or more superacid species.

The superacid composition can also contain an optional corrosion inhibitor. Many nitrogen-containing compounds can serve as corrosion inhibitors including hexamine, benzotriazole, phenylenediamine, dimethylethanolamine, and polyaniline. Further examples of corrosion inhibitors include imines, chromates, and quaternary ammonium silicates. Those skilled in the art will recognize the superacid composition is not limited to any specific corrosion inhibitor. In one embodiment, the superacid composition contains from about 0.001 to about 5% by weight of one or more corrosion inhibitors. In another embodiment, the superacid composition contains from about 0.01 to about 2% by weight of one or more corrosion inhibitors.

As discussed above, mixtures of sulfuric acid and hydrogen peroxide have a tendency to oxidize metal structures contained in semiconductor structures including metal gate structures. Oxidants have a high thermodynamic potential to undergo a reducing reaction to form a more reduced species. The thermodynamic oxidizing ability of an oxidant can be measured by the standard electrode potential for a reduction half-reaction. For example, the half-reaction for the reduction of aqueous hydrogen peroxide to water is +1.78 mV.

In order to guard against the oxidation of metal structures in a semiconductor structure, the superacid composition can be formed to exclude species that have a propensity to oxidize metal structures in the semiconductor structure when included in the superacid composition. As such, the superacid composition can be formulated to not have a propensity to oxidize metal or metal-structures present in the semiconductor structure. As defined herein, a non-corrosive superacid composition does not oxidize more than about 5% of the metal atoms present in the semiconductor structure under conditions needed to remove the resist. In one embodiment, the superacid composition does not contain a species that has a standard electrode potential for reduction half-reaction to a more reduced species greater than +1.5 mV. In another embodiment, the superacid composition does not contain a species that has a standard electrode potential for reduction half-reaction to a more reduce species greater than about +0.5 mV. In yet another embodiment, the superacid composition does not contain a species that has a standard electrode potential for reduction half-reaction to a more reduced species greater than about 0 mV.

Titanium, titanium nitrides and tungsten are increasingly common materials for the formation of metal gates and other metal structures in semiconductor structures and devices. Structures made from pure titanium are resistant to oxidation to form TiO₂. The oxidation of Ti to TiO₂ is thermodynamically favorable; however, titanium forms a passive layer that makes the oxidation of the bulk mass of titanium structures very slow on a kinetic basis. However, titanium nitrides become increasing susceptible to oxidation as the mole fraction of nitrogen in the titanium nitride increases. Tungsten is susceptible to corrosion with oxidizing agents. In one embodiment, the superacid composition does not contain a species capable of oxidizing or corroding titanium nitride structures and/or tungsten structures in a semiconductor structure that is contacted with the superacid composition.

Silicon oxide is commonly used as a dielectric material in semiconductor structures and/or devices. Chemical species that are capable of dissociating to form fluoride ions and/or transferring a fluorine directly to silicon oxide can be excluded from the superacid composition described herein. Chemical species that are capable of liberating fluoride include species that contain a fluorine atom bonded to a heteroatom other than carbon such as sulfur. In one embodiment, the superacid composition does not contain a chemical species having a fluorine-sulfur bond.

To guard against the breakdown of silicon oxide due to exposure to fluorine atoms or fluoride ions, the superacid composition can be prepared without chemical species that contain fluorine. In particular, hydrogen fluoride, antimony pentafluoride and fluorosulphonic acid have a propensity to degrade silicon dioxide. Therefore, in one embodiment, the superacid composition does not contain hydrogen fluoride, antimony pentafluoride and fluorosulphonic. In another embodiment, the superacid composition can contain a chemical species having a fluorine-carbon bond, such as trifluoromethanesulfonic acid.

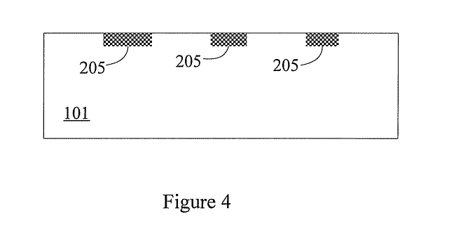

The superacid compositions described herein can be employed during wet stripping procedures to remove a resist and a resist containing carbonized materials. The superacid compositions described herein are capable of completely or substantially removing a resist from the surface of a semiconductor structure without leaving residues including contaminant particles that can be present within the resist and inorganic carbon materials. As shown in FIG. 4, contact of the structure shown in FIG. 2 with the superacid composition results in the removal of resist 105.

Any metal or metal-containing structures on surface of the semiconductor structure underlying the resist should not be etched or oxidized due to exposure to the superacid composition. Contact with the superacid composition results in the dissolution of the resist through means of a chemical reaction between organic and inorganic carbon materials in the resist and the superacid species contained in the superacid composition. As such, lifting and/or peeling of the resist that can result in redeposition of the resist can be avoided.

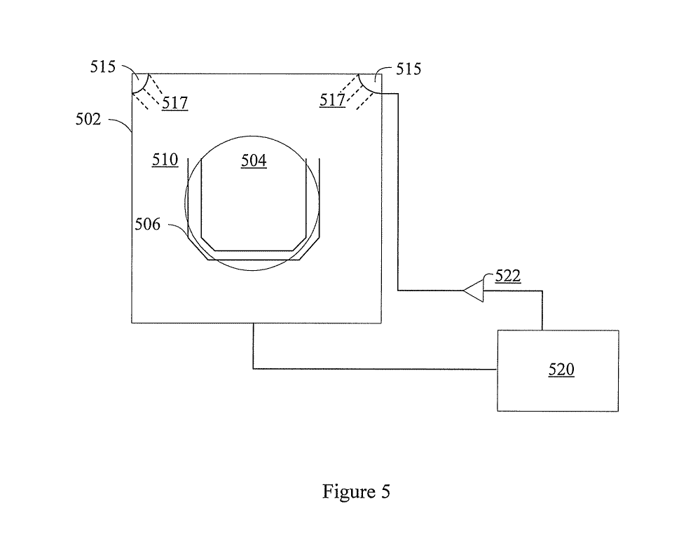

Wet stripping a resist containing inorganic carbonized material from a semiconductor structure can be accomplished by dipping or immersing a semiconductor structure in a tank having a volume of the superacid composition therein. The semiconductor structure can be part of wafer upon which such semiconductor structures are built. An apparatus for employing the superacid composition is described with reference to FIG. 5. In FIG. 5, a tank 502 for stripping a resist is shown from a side perspective. A wafer 504 is present in the interior of tank 502 and positioned by means of a wafer holder 506.

A volume of the superacid composition 510, as described herein, is present in the interior of the tank 502. The superacid composition 510 can be made to recirculate. Recirculation can assist in removing particulate material from the superacid composition as well as provide kinetic energy to assist in the dissolution of the resist from the surface of the wafer 504. As an example, spray nozzles 515 can be provided in the interior of tank 502 to provide a spray 517 of the superacid composition 510. The spray 517 is supplied by a feed of the superacid composition 510 from a reservoir tank 520 by means of a pump 522.

The superacid composition can be supplied at room temperature or the superacid composition can be optionally heated. In one embodiment, the temperature of the superacid composition is from about 15 to about 160° C. In another embodiment, the temperature of the superacid composition is from about 15 to about 140° C. In yet another embodiment, the temperature of the superacid composition is from about 15 to about 120° C.

The wafer 504 having the semiconductor structures covered with a resist has a total contact time with the superacid composition for up to several minutes. In one embodiment, the resist is contacted with the superacid composition from about 5 seconds to about 60 minutes. In another embodiment, the resist is contacted with the superacid composition from about 15 seconds to about 20 minutes. In yet another embodiment, the resist is contacted with the superacid composition from about 1 minute to about 10 minutes.

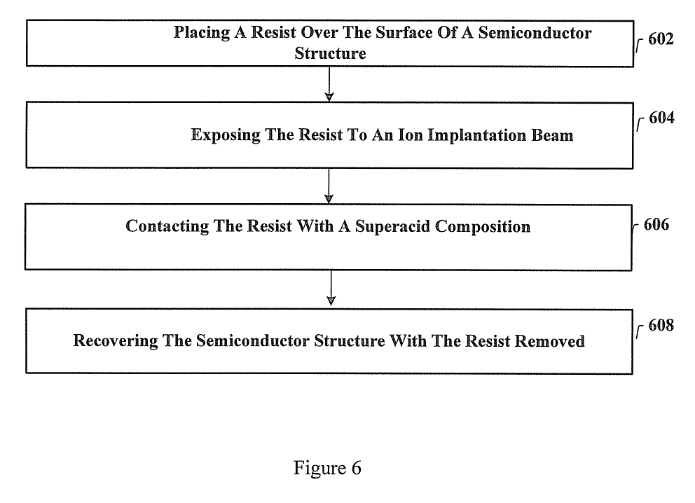

In order to fully describe the innovations disclosed herein, acts for removing a resist having inorganic carbonized material will be described with reference to FIG. 6. In act 602, a resist is placed over a semiconductor structure such that a portion of the surface of the semiconductor structure is protected by the resist and a portion of the semiconductor structure remains accessible. In act 604, the resist is exposed to an ion implantation beam, where the ion implantation beam introduces impurities into the regions of the semiconductor structure that are accessible and not covered by the resist. Exposure of the resist to the ion implantation beam transforms at least a portion of the material forming the resist to an inorganic carbonized material. In act 606, the resist is contacted with a superacid composition containing a superacid species such that the resist having carbonized material is removed from the semiconductor structure. In act 608, a semiconductor structure having substantially all resist material removed including inorganic carbonized material is recovered.

With respect to any figure or numerical range for a given characteristic, a figure or a parameter from one range may be combined with another figure or a parameter from a different range for the same characteristic to generate a numerical range.

Other than in the operating examples, or where otherwise indicated, all numbers, values and/or expressions referring to quantities of ingredients, reaction conditions, etc., used in the specification and claims are to be understood as modified in all instances by the term “about.”

While certain embodiments have been described, these embodiments have been presented by way of example only, and are not intended to limit the scope of the inventions. Indeed, the novel methods and devices described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. A method for removing a carbonized material and/or an organic material from a semiconductor structure, comprising:
contacting the semiconductor structure with a superacid composition comprising a superacid species.

2. The method of claim 1, with the proviso that at least a portion of the semiconductor structure contains or is covered by an inorganic carbonized material containing inorganic carbon-carbon bonds.

3. The method of claim 1, with the proviso that the semiconductor structure is not subjected to an ashing process prior to contact with the superacid composition.

4. The method of claim 1, with the proviso that the superacid composition does not comprise a molecule that dissociates to form free fluoride ion or transfers fluorine to materials forming the semiconductor structure.

5. The method of claim 1, wherein the superacid composition comprises one or more selected from the group consisting of trifluoromethanesulfonic acid, a mixture of antimony pentafluoride and fluorosulfonic acid, a mixture of antimony pentafluoride and hydrofluoric acid, carborane acid, and fluorosulfonic acid.

6. The method of claim 4, wherein the superacid composition comprises one or more selected from the group consisting of trifluoromethanesulfonic acid and carborane acid.

7. The method of claim 1, wherein the superacid composition has a Hammett acidity function of less than about −12.

8. The method of claim 1, wherein the inorganic carbonized material is one or more selected from the group consisting of graphite, fullerene, graphene, carbon nano tube and micro crystallized carbon.

9. The method of claim 1, wherein the superacid composition further comprises at least one corrosion inhibitor.

10. A method for making a semiconductor structure, comprising:
placing a resist over the surface of the semiconductor structure, wherein the resist covers a portion of the semiconductor structure; and
contacting the resist with a superacid composition comprising a superacid species to remove the resist from the semiconductor structure.

11. The method of claim 10, with the proviso that
the resist covers a portion of the semiconductor structure is exposed to an ion beam, where ions are implanted into regions of the semiconductor structure not covered by the resist; and
wherein the resist comprises an organic material prior to performance of ion implantation, and
the resist is converted by exposure to the ion beam to comprise an inorganic carbonized material containing inorganic carbon-carbon bonds.

12. The method of claim 10, with the proviso that the semiconductor structure is not subjected to an ashing process prior to contact with the superacid composition.

13. The method of claim 10, wherein the semiconductor structure contains metal or metal-containing structures, and with the proviso that the superacid composition does not have a propensity to oxidize the metal or metal-containing structures in the semiconductor structure.

14. The method of claim 10, wherein the superacid composition comprises one or more selected from the group consisting of trifluoromethanesulfonic acid, a mixture of antimony pentafluoride and fluorosulfonic acid, a mixture of antimony pentafluoride and hydrofluoric acid, carborane acid, and fluorosulfonic acid.

15. The method of claim 10, wherein the superacid composition has a Hammett acidity function of less than about −12.

16. The method of claim 10, wherein the ions are implanted on the semiconductor structure at a concentration greater than about 1×10¹⁴ atoms/cm².

17. The method of claim 10, wherein the semiconductor structure comprises one or more selected from the group consisting of Ti, Zr, Hf, Nb, Ta, Mo, W, Mn, Fe, Ru, Co, Ni, Pd, Pt, La, Er, Al, Ga, Ge, In, Mg, Y, and alloys thereof.

18. The method of claim 10, wherein the inorganic carbonized material is one or more selected from the group consisting of graphite, fullerene, graphene, carbon nano tube and micro crystallized carbon.

19. The method of claim 10, wherein the superacid composition further comprises at least one corrosion inhibitor.

20. An apparatus for removing a carbonized material from a semiconductor structure comprising:
a tank for holding a volume of a superacid composition therein, the superacid composition comprising a superacid species;
a holder for holding the semiconductor structure within the interior of the tank, and
the semiconductor structure having a carbonized material thereon is present in the container or the holder, where the superacid composition is in contact with the semiconductor structure.

21. The apparatus of claim 20, wherein the superacid composition comprises one or more selected from the group consisting of trifluoromethanesulfonic acid, a mixture of antimony pentafluoride and fluorosulfonic acid, a mixture of antimony pentafluoride and hydrofluoric acid, carborane acid, and fluorosulfonic acid.

22. The apparatus of claim 20, wherein the superacid composition has a Hammett acidity function of less than about −12.

23. An apparatus for removing a carbonized material from a semiconductor structure comprising:
a nozzle for dispensing a superacid composition to the semiconductor structure, wherein the superacid composition comprising a superacid species;
a holder for holding the semiconductor structure, and
the semiconductor structure having a carbonized material thereon is present in the container or the holder, where the superacid composition is in contact with the semiconductor structure.

24. The apparatus of claim 23, wherein the superacid composition comprises one or more selected from the group consisting of trifluoromethanesulfonic acid, a mixture of antimony pentafluoride and fluorosulfonic acid, a mixture of antimony pentafluoride and hydrofluoric acid, carborane acid, and fluorosulfonic acid.

25. The apparatus of claim 23, wherein the superacid composition has a Hammett acidity function of less than about −12.

26. A chemical for removing a carbonized material and/or an organic material from a semiconductor structure comprising a superacid species.

27. The chemical of claim 26, with the proviso that at least a portion of the semiconductor structure contains or is covered by an inorganic carbonized material containing inorganic carbon-carbon bonds.

28. The chemical of claim 26, with the proviso that the semiconductor structure is not subjected to an ashing process.

29. The chemical of claim 26, with the proviso that the chemical does not comprise a molecule that dissociates to form free fluoride ion or transfers fluorine to materials forming the semiconductor structure.

30. The chemical of claim 26, wherein the chemical comprises one or more selected from the group consisting of trifluoromethanesulfonic acid, a mixture of antimony pentafluoride and fluorosulfonic acid, a mixture of antimony pentafluoride and hydrofluoric acid, carborane acid, and fluorosulfonic acid.

31. The chemical of claim 29, wherein the chemical comprises one or more selected from the group consisting of trifluoromethanesulfonic acid and carborane acid.

32. The chemical of claim 26, wherein the chemical has a Hammett acidity function of less than about −12.

33. The chemical of claim 26, wherein the inorganic carbonized material is one or more selected from the group consisting of graphite, fullerene, graphene, carbon nano tube and micro crystallized carbon.

34. The chemical of claim 26, wherein the chemical further comprises at least one corrosion inhibitor.

35. The chemical of claim 26, with the proviso that the resist covers a portion of the semiconductor structure is exposed to an ion beam.
